SEQUENCE LISTING (10 sequences)

SEQ ID NO: 1 - protein, 5 aa, unidentified
SHWVH

SEQ ID NO: 2 - protein, 17 aa, unidentified
EIDPNNGRTNFNEKFKR

SEQ ID NO: 3 - protein, 7 aa, unidentified
ARSSLAY

SEQ ID NO: 4 - protein, 16 aa, unidentified
KSSQSLLDSDGKTYLH

SEQ ID NO: 5 - protein, 7 aa, unidentified
LVSKLDS

SEQ ID NO: 6 - protein, 7 aa, unidentified
WQNTHFP

SEQ ID NO: 7 - protein, 114 aa, unidentified
QVQLQQPGAELVKPGASVRLSCKASGYTFSSHWVHWVRQRPGQGLEWIGEIDPNNGRTNFNEKFKRKATLTVDKSSSTAYMQYSSLTSEDSAVYYCARSSLAYWGQGTLVTVSV

SEQ ID NO: 8 - protein, 112 aa, unidentified
DVVVTQTPLTLSVTIGHPASISCKSSQSLLDSDGKTYLHWFLQRPGQSPKRLIYLVSKLDSGVPDRFTGSGSGTDFTLKISRVEAEDLGVYYCWQNTHFPWTFGGGTKLEIK

SEQ ID NO: 9 - protein, 444 aa, unidentified
QVQLQQPGAELVKPGASVRLSCKASGYTFSSHWVHWVRQRPGQGLEWIGEIDPNNGRTNFNEKFKRKATLTVDKSSSTAYMQYSSLTSEDSAVYYCARSSLAYWGQGTLVTVSVAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 219 aa, unidentified
DVVVTQTPLTLSVTIGHPASISCKSSQSLLDSDGKTYLHWFLQRPGQSPKRLIYLVSKLDSGVPDRFTGSGSGTDFTLKISRVEAEDLGVYYCWQNTHFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2026/0298936 A1
(43) Pub. Date: Oct. 1, 2026

(54) Anti-CLDN18_2 Antibody and an In Vitro Diagnostic Kit

(71) Applicant: Jiangsu Aosaikang Biopharmaceutical Co., Ltd., Nanjing, Jiangsu (CN)

(72) Inventors: Yong WEN, Nanjing (CN); Guwei HOU, Nanjing (CN); Song FU, Nanjing (CN); Jiye SUN, Nanjing (CN)

(73) Assignee: Jiangsu Aosaikang Biopharmaceutical Co., Ltd., Nanjing, Jiangsu (CN)

(21) Appl. No.: 19/490,843

(22) PCT Filed: Jun. 7, 2024
(86) PCT No.: PCT/IB2024/000278

§ 371 Date: Dec. 5, 2025

(30) Foreign Application Priority Data
Jun. 7, 2023 (CN) 202310675165.8

(51) Int. Cl. G01N 33/575; A61K 39/00; C07K 16/28; G01N 33/58

(52) U.S. Cl. CPC G01N 33/5758; C07K 16/28; G01N 33/581; A61K 2039/505; C07K 2317/565; C07K 2317/92; G01N 2333/705; G01N 2333/908

(57) ABSTRACT

The present disclosure relates to kits comprising an anti claudin 18.2 antibody that are useful for assessing expression claudin 18.2 expression in a biological sample obtained from a human, e.g., a human patient having cancer.

### CROSS-REFERENCE TO RELATED APPLICATION

The present application claims priority from Chinese Patent Application No. 202310675165.8, filed on Jun. 7, 2023, the content of which is incorporated by reference herein in its entirety.

### SEQUENCE LISTING

The present application contains a Sequence Listing which has been submitted electronically in XML format and is hereby incorporated by reference in its entirety. The copy of the Sequence Listing, created on Jun. 4, 2024, is named 025471.WO017.xml and is 10,134 bytes in size.

### BACKGROUND

Gastric cancer is one of the most common cancers worldwide. In 2020 alone, there were 479,000 new cases and 374,000 deaths from gastric cancer in China. Even though the 5-year survival rate of gastric cancer in China increased from 27.4% in 2003 to 35.1% in 2015, it is still significantly lower than in some other countries/regions (Chinese Guidelines for Screening, Early Diagnosis and Treatment of Gastric Cancer (2022), National Cancer Center of China). These statistics indicate an urgent need for improved diagnosis and treatment of gastric cancer.

Antibody therapeutics have been widely used in treatment of cancers. Antibody-based targeted therapy has higher specificity and fewer side effects compared to traditional cancer therapy such as chemotherapy. Claudin 18.2 (CLDN18.2 or CLDN18_2) has been found to be a promising antibody target for the treatment of gastric and esophageal cancer (Singh et al., J Hematol Oncol. (2017) 10(1):105). It is also being investigated as an antibody target for pancreatic cancer. Currently, several anti-CLDN18.2 monoclonal antibodies are under clinical development, including zolbetuximab (chimeric antibody) and ASKB589 (humanized antibody).

CLDN18.2 belongs to a family of proteins (claudins) that constitute tight junctions between cells. Tight junctions determine the permeability of epithelial cells and play a role in blocking diffusion of cell surface proteins and lipids. Claudin 18, a member of the family, is encoded by the claudin 18 gene. The human claudin 18 gene has two different exons 1, which undergo alternative splicing when transcribed and ultimately generate two isoforms, claudin 18.1 and claudin 18.2, which differ in their N-terminal sequences. The expression of claudin 18.1 and claudin 18.2 in humans is tissue-specific. Claudin 18.1 is mainly expressed in lung tissue but not in gastric tissue or gastric cancer. Claudin 18.2 is a tight-junction molecule predominantly found in the normal gastric epithelium and becomes accessible on the surface of cancerous cells for antibody targeting during malignant transformation. Due to its expression in various epithelial cancers and its highly restricted expression pattern in normal tissues, claudin 18.2 has become a target for developing treatment of epithelial cancers.

To improve efficacy of cancer therapeutics that target claudin 18.2, there is a need to assess claudin 18.2 expression in cancer patients in order to better guide the use of these therapeutics.

### SUMMARY

In the first aspect, the present disclosure provides an anti-CLDN18.2 antibody or an antigen-binding fragment thereof. The anti-CLDN18.2 antibody includes: (1) an antibody heavy chain CDR1 with an amino acid sequence shown in SEQ ID NO:1, (2) an antibody heavy chain CDR2 with an amino acid sequence shown in SEQ ID NO:2, (3) an antibody heavy chain CDR3 with an amino acid sequence shown in SEQ ID NO:3, (4) an antibody light chain CDR1 with an amino acid sequence shown in SEQ ID NO:4, (5) an antibody light chain CDR2 with an amino acid sequence shown in SEQ ID NO:5, and (6) an antibody light chain CDR3 with an amino acid sequence shown in SEQ ID NO:6.

In some embodiments, the anti-CLDN18.2 antibody includes: an antibody heavy chain variable region with an amino acid sequence shown in SEQ ID NO:7 or with a similarity not less than 90% (for example, not less than 90%, or not less than 95%, or not less than 98%, or not less than 99%) to the amino acid sequence shown in SEQ ID NO:7, and an antibody light chain variable region with an amino acid sequence shown in SEQ ID NO:8 or with a similarity not less than 90% (for example, not less than 90%, or not less than 95%, or not less than 98%, or not less than 99%) to the amino acid sequence shown in SEQ ID NO:8.

In some embodiments, the anti-CLDN18.2 antibody is a DS-3 antibody as recorded in the present disclosure.

In some embodiments, the anti-CLDN18.2 antibody includes: an antibody heavy chain with an amino acid sequence shown in SEQ ID NO:9 or with a similarity not less than 90% (for example, not less than 90%, or not less than 95%, or not less than 98%, or not less than 99%) to the amino acid sequence shown in SEQ ID NO:9, and an antibody light chain with an amino acid sequence shown in SEQ ID NO:10 or with a similarity not less than 90% (for example, not less than 90%, or not less than 95%, or not less than 98%, or not less than 99%) to the amino acid sequence shown in SEQ ID NO:10.

In the second aspect, the present disclosure provides an isolated nucleic acid, which encodes the antibody or the antigen-binding fragment thereof as described in the first aspect of the present disclosure.

In the third aspect, the present disclosure provides a recombinant expression vector, which contains the nucleic acid as described in the second aspect of the present disclosure.

In the fourth aspect, the present disclosure provides an in vitro diagnostic kit, which contains the anti-CLDN18.2 antibody or the antigen-binding fragment thereof. The anti-CLDN18.2 antibody includes: (1) an antibody heavy chain CDR1 with an amino acid sequence shown in SEQ ID NO:1, (2) an antibody heavy chain CDR2 with an amino acid sequence shown in SEQ ID NO:2, (3) an antibody heavy chain CDR3 with an amino acid sequence shown in SEQ ID NO:3, (4) an antibody light chain CDR1 with an amino acid sequence shown in SEQ ID NO:4, (5) an antibody light chain CDR2 with an amino acid sequence shown in SEQ ID NO:5, and (6) an antibody light chain CDR3 with an amino acid sequence shown in SEQ ID NO:6.

In some embodiments, the anti-CLDN18.2 antibody includes: an antibody heavy chain variable region with an amino acid sequence shown in SEQ ID NO:7 or with a similarity not less than 90% (for example, not less than 90%, or not less than 95%, or not less than 98%, or not less than 99%) to the amino acid sequence shown in SEQ ID NO:7, and an antibody light chain variable region with an amino acid sequence shown in SEQ ID NO:8 or with a similarity not less than 90% (for example, not less than 90%, or not less than 95%, or not less than 98%, or not less than 99%) to the amino acid sequence shown in SEQ ID NO:8.

In some embodiments, the anti-CLDN18.2 antibody includes: an antibody heavy chain with an amino acid sequence shown in SEQ ID NO:9 or with a similarity not less than 90% (for example, not less than 90%, or not less than 95%, or not less than 98%, or not less than 99%) to the amino acid sequence shown in SEQ ID NO:9, and an antibody light chain with an amino acid sequence shown in SEQ ID NO:10 or with a similarity not less than 90% (for example, not less than 90%, or not less than 95%, or not less than 98%, or not less than 99%) to the amino acid sequence shown in SEQ ID NO:10.

In some embodiments, the anti-CLDN18.2 antibody or the antigen-binding fragment thereof is coupled with at least one detectable marker.

In some embodiments, the anti-CLDN18.2 antibody preparation of said antibody diagnostic kit comprises said antibody anti-CLDN18.2 antibody, phosphate buffer, sodium chloride, surfactant, bovine serum albumin, and a preservative, and has a pH between 7-8.

In some embodiments, the anti-CLDN18.2 antibody preparation of said antibody diagnostic kit comprises:
0.05-0.2 μg/mL of said antibody anti-CLDN18.2 antibody
0.05-0.2 M phosphate buffer,
0.05-0.2 M sodium chloride,
a surfactant,
0.5-2% (w/v) bovine serum albumin, and
a preservative, and has a pH between 7-8.

In some embodiments, the anti-CLDN18.2 antibody preparation of said antibody diagnostic kit comprises said antibody anti-CLDN18 comprises:
0.1 μg/mL of said antibody anti-CLDN18.2 antibody
0.1 M phosphate buffer,
0.05-0.0.6 M sodium chloride,
a surfactant, which is 4-(1,1,3,3-tetramethylbutyl) phenyl-polyethylene glycol, at a concentration of approximately 0.5% (v/v),
1.0% (w/v) bovine serum albumin, and
a preservative, and
has a pH between 7.2-7.5

In some embodiments, the anti-CLDN18.2 antibody preparation of said antibody diagnostic kit comprises said antibody anti-CLDN18 comprises:
0.1 μg/mL of said antibody anti-CLDN18.2 antibody
0.1 M phosphate buffer,
0.05-0.0.6 M sodium chloride,
a surfactant, which is Triton X-100, at a concentration of approximately 0.5% (v/v),
1.0% (w/v) bovine serum albumin, and
a preservative, which is ProClin™ 300, at a concentration of approximately 0.05% (v/v), and has a pH between 7.2-7.5

In the fifth aspect, the present disclosure provides a method for detecting a sample using the in vitro diagnostic kit as described in the fifth aspect of the present disclosure.

In some embodiments, the present method detects claudin 18.2 (CLDN18.2) in a biopsy obtained from a human (e.g., a human cancer patient); the method comprises contacting the biopsy with an anti-CLDN18.2 antibody or an antigen-binding fragment herein and detecting the binding of the antibody or antigen-binding fragment to the biopsy, wherein the presence of binding is indicative of the presence of CLDN18.2 in the biopsy.

In some embodiments, the present method diagnoses cancer in a human; the method comprises contacting a biopsy from the human with an anti-CLDN18.2 antibody or an antigen-binding fragment herein and detecting the binding of the antibody or antigen-binding fragment to the biopsy, wherein the presence of binding is indicative of the presence of cancer in the human.

In some embodiments, a method for detecting a sample includes: (i) fixing the sample on a solid support; (n) contacting the sample with the anti-CLDN18.2 antibody or the antigen-binding fragment thereof included in the in vitro diagnostic kit as described in the fifth aspect of the present disclosure; (iii) contacting the anti-CLDN18.2 antibody or the antigen-binding fragment thereof with a detectable marker; and (iv) determining a binding level of the anti-CLDN18.2 antibody or the antigen-binding fragment thereof with the sample. The detectable marker is used for marking the anti-CLDN18.2 antibody or the antigen-binding fragment thereof.

In some embodiments, a method for detecting a sample is provided, including: (i) fixing the sample on a solid support; (n) contacting the sample with the anti-CLDN18.2 antibody or the antigen-binding fragment thereof included in the in vitro diagnostic kit as described in the fifth aspect of the present disclosure; and (iii) determining a binding level of the antibody or the antigen-binding fragment thereof with the sample through a detectable marker. The anti-CLDN18.2 antibody or the antigen-binding fragment thereof is coupled with at least one detectable marker.

In some embodiments, the detectable marker is horseradish peroxidase.

In some embodiments, the sample is a tissue section.

In some embodiments, the tissue is selected from the pancreas, esophagus, ovaries, lungs, stomach, bronchi, mammary glands, or ear, nose, and throat.

In some embodiments, the tissue is selected from a tumor of gastric cancer, pancreatic cancer, biliary tract cancer, or esophagus cancer.

In some embodiments, the tissue is selected from a tumor located at pancreas, esophagus, ovaries, lungs, stomach, bronchi, mammary glands, colon, biliary tract, or throat.

In some embodiments, the solid support is a glass slide.

In the sixth aspect, the present disclosure provides a method of treating cancer in a human in need thereof; the method comprises detecting the presence of CLDN18.2 in a biopsy obtained from the human with the present detection method and treating the patient with an anti-CLDN18.2 antibody therapeutic if CLDN18.2 is found to be present in the biopsy.

Those skilled in the art may easily determine a CDR sequence based on the sequence of a heavy or light chain variable region. To perform CDR definition on an antibody variable region, an antibody numbering scheme and a definition scheme are required. The antibody numbering scheme includes but is not limited to: IMGT, Chothia, Kabat and Martin (extended version of Chothia), and the definition scheme includes but is not limited to Chothia, Kabat, IMGT and Contact.

The present disclosure provides an anti-CLDN18.2 antibody or an antigen-binding fragment thereof, and an in vitro diagnostic kit containing the anti-CLDN18.2 antibody or the antigen-binding fragment thereof. The antibody or the antigen-binding fragment thereof has high binding activity with CLDN18.2 protein. The in vitro diagnostic kit containing the antibody or the antigen-binding fragment thereof has simplicity and convenience in operation, high detection sensitivity and storage stability.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIGS. 1A and 1B show the comparison of the performance of antibody DS-3 with another antibody (a mouse antibody) in the staining of gastric cancer tissue sections. In the tissue sections shown, the DS-3 antibody showed more significant claudin 18.2 coloration compared with the mouse anti-CLDN18.2 antibody (lot number PW03-33) (FIG. 1B).

FIGS. 1C and 1D shows the comparison of the DS-3 antibody with a rabbit antibody.

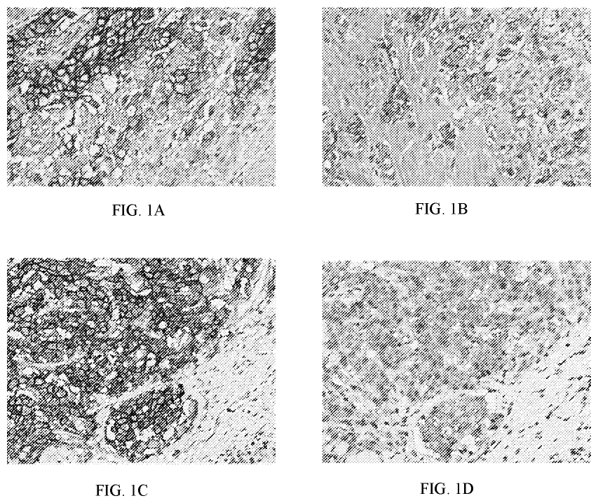

In human gastric cancer tissue sections, the DS-3 antibody showed more significant color development of claudin 18.2 (FIG. 1C) compared with the rabbit-derived anti-CLDN18.2 antibody (AbCam, Clone No. EPR19202-244) (FIG. 1D).

### DETAILED DESCRIPTION

The present disclosure provides methods for detecting the presence of claudin 18.2 expression in human patients (e.g., e.g., cancer patients) and companion diagnostic kits for treating cancer patients with anti-claudin 18.2 antibody therapeutics. Companion diagnostics may be used in selecting or excluding patients for treatment with a specific therapy based on biological characteristics that determine likely responders and likely non-responders to the therapy. The present detection methods and kits can help guide anti-CLDN18.2 cancer treatment and improve the efficacy of such treatment.

The CLDN18.2-detecting antibodies or antigen-binding fragments provided herein are superiorly sensitive and specific for detecting CLDN18.2 in tissue samples. These antibodies and fragments may be used, for example, in immunohistochemistry staining of tissue sections obtained from patients (e.g., paraffin sections of biopsies from patients with various cancers such as gastric cancer, gastroesophageal junction cancer, and other epithelial cancers).

In the present disclosure, the term “homology” or “identity” with respect to sequence alignment refers to the proportion (e.g., percentage) of similar or identical amino acids, respectively, between a query sequence and a reference sequence over the entire length of the reference sequence. Similarity in amino acid sequence alignment includes two residues at corresponding positions having similar characteristics, e.g., size, charge, and hydrophilicity of side chain groups, as well as identical residues. Sequence alignment methods are common technical knowledge in the art and may be performed by, e.g., the BLAST® program.

In the present disclosure, the terms “claudin 18” and “CLDN18” are used interchangeably herein. The terms “claudin 18.1”, “CLDN18.1” and “CLDN18_1” are used interchangeably herein, and the terms “claudin 18.2”, “CLDN18.2” and “CLDN18_2” are also used interchangeably herein.

In the present disclosure, “mass volume percentage” or “% (w/v)” or “% w/v” is a representation of mass volume concentration, representing the grams of solute contained in every 100 ml of solution, for example, 20% (w/v) represents the presence of 20 g solute per 100 ml of solution.

### I. Kits for Assessing Claudin 18.2 Expression In Vitro

The present disclosure provides kits for assessing in vitro the expression of claudin 18.2 in a biological sample obtained from a human, e.g., a human patient having or suspected of having cancer. These kits also may also be referred to herein as in vitro diagnostic kits and include the anti-CLDN18.2 antibody or antigen-binding fragment and detectable agents linked, or to be linked, to the antibody or fragment. The kits may also include a calibrator and/or a control. The kits may include instructions for how to perform in vitro detection of CLDN18.2 in a biological sample, and may additionally include instructions for informing clinicians how to use the CLDN18.2 expression information to guide treatment. The kits may be used in the process of monitoring hyperplasia, diagnosing and prognosing cancer, monitoring disease progression and treatment, and evaluating disease conditions in a human patient. The kits can be used alone or in combination with other instruments and systems.

### A. Anti-CLDN18.2 Antibodies

The CLDN18.2-detecting agent is an anti-CLDN18.2 antibody or an antigen-binding fragment thereof. The antibody or fragment may comprise one, two, three, four, five, or six of the complementarity-determining regions (CDRs), the heavy chain variable region (VH) and/or the light chain variable region (VL), or the heavy chain and/or the light chain, of the anti-CLDN18.2 DS-3 antibody described herein.

A number of CDR delineations are known in the art and are encompassed herein. A skilled person in the art can readily determine a CDR of a given delineation based on the heavy or light chain variable region sequence. The “Kabat” CDRs are based on sequence variability and are commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, J Mol Biol. (1987) 196:901-917). The “AbM” CDRs represent a compromise between the Kabat CDRs and Chothia structural loops, and are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid numbers in antibodies refer to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 | H31-H35B | H26-H35B | H26-H32 | H30-H35B |
| (Kabat nos.) | | | | |
| VH-CDR1 | H31-H35 | H26-H35 | H26-H32 | H30-H35 |
| (Chothia nos.) | | | | |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

In some embodiments, the anti-CLDN18.2 antibody or fragment herein comprises heavy chain complementarity-determining regions (CDR) 1-3 set forth in SEQ ID NOs:1-3, respectively, and/or light chain CDR 1-3 set forth in SEQ ID NOs:4-6, respectively.

In some embodiments, the anti-CLDN18.2 antibody or fragment herein comprises a VH comprising SEQ ID NO:7 or an amino acid sequence at least 90% identical thereto, and/or a VL comprising SEQ ID NO:8 or an amino acid sequence at least 90% identical thereto. In further embodiments, the antibody or fragment comprises a VH with an amino acid sequence of SEQ ID NO:7 and a VL with an amino acid sequence of SEQ ID NO:8. In certain embodiments, the antibody may be of a mouse IgG1, IgG2, IgG3, or IgG3 isotype.

In some embodiments, the anti-CLDN18.2 antibody or fragment herein comprises a heavy chain comprising SEQ ID NO:9 or an amino acid sequence at least 90% identical thereto, and/or a light chain comprising SEQ ID NO:10 or an amino acid sequence at least 90% identical thereto. In further embodiments, the antibody is the DS-3 antibody, with heavy and light chain sequences of SEQ ID NOs:9 and 10, respectively.

### B. Detectable Markers

In some embodiments, the kit of the present disclosure provides a detectable marker. For example, the present of CLDN18.2 in a sample can be detected by an anti-CLDN18.2 antibody or fragment herein labeled with a fluorescent moiety, a metal, or an enzyme. In some embodiments, the detection reagent may be included in or on the tissue collection device (e.g., an antibody or an indicator chemical embedded on a test strip or a glass slide). In other embodiments, the detection reagent is provided separately from the collection device as part of the kit.

In some embodiments, the detectable marker is coupled directly to the anti-CLDN18.2 antibody or fragment for direct detection, or is used together with a secondary reagent (such as an antibody that recognizes the anti-CLDN18.2 antibody or fragment and is labeled with the detectable marker) for indirect detection. In other words, the anti-CLDN18.2 antibody or fragment may be directly or indirectly coupled to the detectable marker.

In some embodiments, the detectable maker is a chromogenic reagent that uses a colored enzyme substrate or a fluorescent agent. Non-limiting examples of chromogenic reagents include horseradish peroxidase or alkaline phosphate, both of which can catalyze a color-producing reaction in the presence of a chromogenic substrate like diaminobenzidine (DAB). Non-limiting examples of fluorescent reagents include fluorescein isothiocyanate (FITC), tetramethylrhodamine isothiocyanate (TRITC), aminomethyl coumarin acetate (AMCA), cyanine5 (Cy5), or Alexa Fluor® fluorochromes. Chromogenic, fluorogenic, and chemiluminescent reagents are widely known and available in the art (e.g., from companies such as Thermo Fisher Scientific, AbCam, etc.).

### C. Antibody Compositions

In some embodiments, the kit provides the antibody or fragment in a dry powder (e.g., lyophilized) form, which can then be reconstituted in an aqueous buffered solution that is optionally included in the kit, to generate an anti-CLDN18.2 antibody or fragment preparation for use. In other embodiments, the kit provides the antibody or fragment in an aqueous buffered solution or preparation.

An aqueous solution may comprise a phosphate buffer, sodium chloride, a surfactant, an albumin (e.g., bovine serum albumin), and a preservative, at a pH of about 7 to 8. In some embodiments, the aqueous solution comprises 0.01-0.5 μg/mL of anti-CLDN18.2 antibody or fragment.

In some embodiments, 0.01-0.5 M phosphate buffer is used in the aqueous solution. The phosphate buffer may be prepared by combining disodium hydrogen phosphate and sodium dihydrogen phosphate; for example, the combination of disodium hydrogen phosphate and sodium dihydrogen phosphate may be used at a ratio of 1:1, 2:1, or 12. The buffer may additionally contain sodium chloride, at, e.g., a concentration of 0.01-0.5 M.

In some embodiments, a surfactant is used in aqueous solution. Non-limiting examples of surfactants include Triton™ X-100 (Sigma Aldrich; CAS No.: 9036-19-5; 4-(1,1,3,3-tetramethylbutyl) phenyl-polyethylene glycol).

In some embodiments, 0.1-5% (w/v) bovine serum albumin is used in aqueous solution. In some embodiments, the aqueous solution is at a pH of about 6-9. In particular embodiments, the anti-CLDN18.2 antibody is at a pH of about 7-8, or in more particular embodiments, the anti-CLDN18.2 antibody is a at pH of about 7-7.5, such as 7.0, 7.1, 7.2, 7.3, 7.4, or 7.5.

In some embodiments, the aqueous solution comprises a preservative or a bacteriostatic agent such as ProClin™ 300, at a concentration of, e.g., about 0.05% (v/v). ProClin™ 300 (Sigma Aldrich) comprises of 3% of 5-chloro-2-methyl-4-isothiazolin-3-one (CMIT) and 2-methyl-4-isothiazolin-3-one (MIT) in a salt-free glycol containing an alkyl carboxylate stabilizer. It has a recommended working pH range as 2.5-8.5.

By way of example, the aqueous solution in which the antibody or fragment is provided or reconstituted comprises 0.05-0.2 μg/mL of anti-CLDN18.2 antibody, 0.05-0.2 M phosphate buffer, 0.05-0.2 M sodium chloride, a surfactant, 0.5-2% (w/v) bovine serum albumin, and a preservative, and the aqueous solution is at a pH of about 7-8. In some embodiments, the aqueous solution comprises 0.1 μg/mL of anti-CLDN18.2 antibody, 0.1 M phosphate buffer, 0.05-0.0.6 M sodium chloride, Triton™ X-100 as a surfactant at a concentration of approximately 0.5% (v/v), 1.0% (w/v) bovine serum albumin, and a preservative, and is at a pH of about 7-7.5, preferably 7.2-7.5. In further embodiments, the aqueous solution comprises 0.1 μg/mL of anti-CLDN18.2 antibody, 0.1 M phosphate buffer, 0.05-0.0.6 M sodium chloride, Triton™ X-100 at a concentration of approximately 0.5% (v/v), 1.0% (w/v) bovine serum albumin, and ProClin™ 300, at a concentration of approximately 0.05% (v/v), and wherein the aqueous solution is at a pH of about 7-7.5, preferably 7.2-7.5.

### D. Other Kit Components

In some embodiments, the kit provides a means or device for collecting a biological sample, such as one or more of a test tube, syringe, a glass slide, a test strip, and a container for storing the biological sample. Any other device known in the art for collecting and processing a biological sample such as a biopsy can also be included.

In some embodiments, the kit can further comprise a device for quantifying or semi-quantifying CLDN18.2 expression in the biological sample. Alternatively, such quantification may be performed with means outside the kit. Non-limiting examples of such means are a microscope, a colorimeter, a spectrometer, an electromagnetic frequency spectrometer (e.g., UV-VIS, IR, or NMR).

The kit may comprise instructions for obtaining, processing, and staining a biological sample obtained from a human patient. In some embodiments, the kit further comprises instructions to compare the level of CLDN18.2 expression in the biological sample to a reference level.

### I. Methods of Use

The present kits and methods may be used to detect (e.g., the presence and/or levels of) CLDN18.2 in a biological sample (e.g., a tissue biopsy) taken from a subject (e.g., a human patient) known to have or suspected of having cancer (e.g., epithelial cancer). In some embodiments, the subject has gastric cancer or gastroesophageal junction cancer. In some other embodiments, the cancer is cancer of the ear, nose, throat, esophagus, stomach, pancreas, biliary tract, liver, colon, ovary, mammary gland, lung, bronchus, or skin.

The biological sample may be taken from a tissue having or suspected of having cancerous cells. For example, the biological sample may be a biopsy taken from a peripheral tissue or an internal tissue. The biological sample may be taken from, for example, the ear, nose, throat, esophagus, stomach, pancreas, biliary tract, liver, colon, ovary, mammary gland, lung, bronchus, or skin. The biological sample may be a liquid biopsy, which may contain cancerous cells (e.g., circulating tumor cells). Examples include plasma, whole blood (e.g., dried blood spot), and serum.

As noted above, claudin 18.2 is a tight-junction molecule predominantly found in nonmalignant gastric epithelium and becomes accessible on the tumor cell surface during malignant transformation. Thus, a subject being screened for or diagnosed with a cancer in accordance with the present methods may express detectable claudin 18.2 on the surface of cancerous cells, in contrast to normal cells of the same tissue. In some embodiments, the cancerous cells express more CLDN18.2 than normal cells by 2-5%, 5-10%, 5-20%, 5-30%, 5-40%, or at least 50% in a human (e.g., an adult, adolescent, or pediatric human patient).

By way of example, the present disclosure provides a method for detecting CLDN18.2 expression in a biological sample obtained from a human patient having cancer comprising: fixing the biological sample on a solid support; contacting the biological sample with an aqueous solution of the kit disclosed herein comprising anti-CLDN18.2 antibody; contacting the biological sample with a signal-generating reagent that allows detection of the anti-CLDN18.2 antibody bound to the solid support; and determining the presence or quantifying the level of CLDN18.2 binding by the antibody.

In some embodiments, a biopsy (e.g., a cancer biopsy) is embedded in paraffin sections for IHC staining of claudin 18.2. In some embodiments, the biopsy is sectioned and fixed on a solid support (e.g., a glass slide) for staining. After staining is completed, the slides may be dehydrated and mounted for generating quality sections using a microtome instrument available in the art. A non-limiting example of such an instrument includes a Dakewe fully-automatic staining and mounting machine. IHC instruments are well known in the art; a non-limiting example is a Leica Bond™ III fully-automatic IHC instrument.

Once a cancer is determined to be CLDN18.2-positive, the patient may be treated with an anti-CLDN18.2 immunotherapy. One such example is immunotherapy using zolbetuximab. Another example is immunotherapy using AKSB589.

### III. Exemplary Embodiments

In order that this invention may be better understood, the following exemplary embodiments are set forth. These embodiments are illustrative and are to be construed as limiting the scope of the invention in any manner.
1. An in vitro diagnostic kit, containing an anti-CLDN18.2 antibody or an antigen-binding fragment thereof, the anti-CLDN18.2 antibody comprising:
   (1) an antibody heavy chain CDR1 with an amino acid sequence shown in SEQ ID NO:1,
   (2) an antibody heavy chain CDR2 with an amino acid sequence shown in SEQ ID NO 2,
   (3) an antibody heavy chain CDR3 with an amino acid sequence shown in SEQ ID NO:3,
   (4) an antibody light chain CDR1 with an amino acid sequence shown in SEQ ID NO:4,
   (5) an antibody light chain CDR2 with an amino acid sequence shown in SEQ ID NO:5, and
   (6) an antibody light chain CDR3 with an amino acid sequence shown in SEQ ID NO:6.
2. The in vitro diagnostic kit according to embodiment 1, wherein the anti-CLDN18.2 antibody comprises:
   an antibody heavy chain variable region with an amino acid sequence shown in SEQ ID NO:7 or with a similarity not less than 90% to the amino acid sequence shown in SEQ ID NO:7, and
   an antibody light chain variable region with an amino acid sequence shown in SEQ ID NO:8 or with a similarity not less than 90% to the amino acid sequence shown in SEQ ID NO:8.
3. The in vitro diagnostic kit according to embodiment 1 or 2, wherein the anti-CLDN18.2 antibody or the antigen-binding fragment thereof is coupled with at least one detectable marker.
4. The in vitro diagnostic kit according to embodiment 1 or 2, wherein the anti-CLDN18.2 antibody preparation comprises said antibody anti-CLDN18.2 antibody, phosphate buffer, sodium chloride, surfactant, bovine serum albumin, and a preservative, and has a pH between 7-8.
5. The in vitro diagnostic kit according to embodiment 1 or 2, wherein the anti-CLDN18.2 antibody preparation comprises:
   0.05-0.2 μg/mL of said antibody anti-CLDN18.2 antibody
   0.05-0.2M phosphate buffer,
   0.05-0.2M sodium chloride,
   surfactant,
   0.5-2% (w/v) bovine serum albumin, and
   a preservative,
   and has a pH between 7-8.
6. The in vitro diagnostic kit according to embodiment 1 or 2, wherein the anti-CLDN18.2 antibody preparation comprises:
   0.1 μg/mL of said antibody anti-CLDN18.2 antibody
   0.1 M phosphate buffer,
   0.05-0.0.6 M sodium chloride,
   a surfactant, which is 4-(1,1,3,3-tetramethylbutyl) phenyl-polyethylene glycol, at a concentration of approximately 0.5% (v/v),
   1.0% (w/v) bovine serum albumin, and
   a preservative,
   and has a pH between 7.2-7.5
7. The in vitro diagnostic kit according to embodiment 1 or 2, wherein the anti-CLDN18.2 antibody preparation comprises:
   0.1 μg/mL of said antibody anti-CLDN18.2 antibody
   0.1 M phosphate buffer,
   0.05-0.0.6 M sodium chloride,
   a surfactant, which is Trixton X-100, at a concentration of approximately 0.5% (v/v),
   1.0% (w/v) bovine serum albumin, and
   a preservative, which is Proclin 300, at a concentration of approximately 0.05% (v/v),
   and has a pH between 7.2-7.5
8. A method for detecting a sample, comprising:
   (i) fixing the sample on a solid support;
   (ii) contacting the sample with the anti-CLDN18.2 antibody or the antigen-binding fragment thereof comprised in the in vitro diagnostic kit according to claim 1 or 2;
   (iii) contacting the anti-CLDN18.2 antibody or the antigen-binding fragment thereof with a detectable marker; and
   (iv) determining a binding level of the anti-CLDN18.2 antibody or the antigen-binding fragment thereof with the sample;
the detectable marker being used for marking the anti-CLDN18.2 antibody or the antigen-binding fragment thereof.
9. A method for detecting a sample, comprising:
   (i) fixing the sample on a solid support;
   (ii) contacting the sample with the anti-CLDN18.2 antibody or the antigen-binding fragment thereof comprised in the in vitro diagnostic kit according to claim 7; and
   (iii) determining a binding level of the antibody or the antigen-binding fragment thereof with the sample through a detectable marker.
10. The method according to embodiment 8 or 9, wherein the detectable marker is horseradish peroxidase.
11. The method according to embodiment 8 or 9, wherein the sample is a tissue section.
12. The method according to embodiment 11, wherein the tissue is selected from the pancreas, esophagus, ovaries, lungs, stomach, bronchi, mammary glands, or ear, nose, and throat.
13. The method according to embodiment 11, wherein the tissue is selected from a tumor located at the pancreas, esophagus, ovaries, lungs, stomach, bronchi, mammary glands, biliary tract, colon, or ear, nose, and throat.
14. The method according to embodiment 11, wherein the tissue is selected from a tumor of the gastric cancer, pancreatic cancer, biliary tract cancer, or esophagus cancer
15. The method according to embodiment 8 or 9, wherein the solid support is a glass slide.
16. A method of detecting claudin 18.2 (CLDN18.2) in a biopsy obtained from a human, comprising:
   contacting the biopsy with an anti-CLDN18.2 antibody or an antigen-binding fragment thereof comprising heavy chain CDR1-3 comprising SEQ ID NOs:1-3, respectively, and light chain CDR1-3 comprising SEQ ID NOs:4-6, respectively; and
   detecting the binding of the antibody or antigen-binding fragment to the biopsy, wherein the presence of binding is indicative of the presence of CLDN18.2 in the biopsy.
17. A method of diagnosing cancer in a human, comprising:
   contacting a biopsy from the human with an anti-CLDN18.2 antibody or an antigen-binding fragment thereof comprising heavy chain CDR1-3 comprising SEQ ID NOs:1-3, respectively, and light chain CDR1-3 comprising SEQ ID NOs:4-6, respectively; and
   detecting the binding of the antibody or antigen-binding fragment to the biopsy, wherein the presence of binding is indicative of the presence of cancer in the human.
18. The method of embodiment 17, wherein the biopsy is taken from a tissue that does not normally express detectable claudin 18.2, optionally wherein the tissue is a tissue other than gastric epithelium.
19. The method of any one of embodiments 16-19, wherein the antibody or antigen-binding fragment comprises
   a heavy chain variable region comprising SEQ ID NO:7 or an amino acid sequence at least 90% identical thereto, and
   a light chain variable region comprising SEQ ID NO:8 or an amino acid sequence at least 90% identical thereto.
20. The method of any one of embodiments 16-19, wherein the antibody or antigen-binding fragment comprises
   a heavy chain comprising SEQ ID NO:9 or an amino acid sequence at least 90% identical thereto, and
   a light chain comprising SEQ ID NO:10 or an amino acid sequence at least 90% identical thereto.
21. The method of any one of embodiments 16-20, wherein the antibody or antigen-binding fragment is linked directly or indirectly to a detectable marker.
22. The method of embodiment 21, wherein the detectable marker is horseradish peroxidase.
23. The method of any one of embodiments 16-22, wherein the biopsy is obtained from the ear, nose, throat, esophagus, stomach, pancreas, liver, biliary tract, colon, lung, bronchus, breast, or ovary.
24. The method of any one of embodiments 16-23, wherein the biopsy is sectioned and fixed on a solid support before being contacted with the anti-CLDN18.2 antibody or fragment, optionally wherein the solid support is a glass slide.
25. A kit comprising an anti-CLDN18.2 antibody or an antigen-binding fragment thereof, wherein the antibody or antigen-binding fragment comprises heavy chain CDR1-3 comprising SEQ ID NOs:1-3, respectively, and light chain CDR1-3 comprising SEQ ID NOs:4-6, respectively.
26. The kit of embodiment 25, wherein the antibody or antigen-binding fragment comprises
   a heavy chain variable region comprising SEQ ID NO:7 or an amino acid sequence at least 90% identical thereto, and
   a light chain variable region comprising SEQ ID NO:8 or an amino acid sequence at least 90% identical thereto.
27. The kit of embodiment 25, wherein the antibody or antigen-binding fragment comprises
   a heavy chain comprising SEQ ID NO:9 or an amino acid sequence at least 90% identical thereto, and
   a light chain comprising SEQ ID NO:10 or an amino acid sequence at least 90% identical thereto.
28. The kit of any one of embodiments 25-27, wherein the antibody or antigen-binding fragment is present in a phosphate-buffered solution comprising sodium chloride, a surfactant, bovine serum albumin, and a preservative, with a pH of 7-8.
29. The kit of any one of embodiments 25-27, wherein the kit comprises
   the antibody or antigen-binding fragment in a lyophilized form and
   a reconstitution phosphate-buffered solution comprising sodium chloride, a surfactant, bovine serum albumin, and a preservative, with a pH of 7-8.
30. A method of treating cancer in a human in need thereof, comprising:
   detecting the presence of CLDN18.2 in a biopsy obtained from the human with a method of any one of embodiments 15-24, and
   treating the patient with an anti-CLDN18.2 antibody therapeutic if CLDN18.2 is found to be present in the biopsy.
31. The method of embodiment 15, wherein the biopsy is taken from a tissue that does not normally express detectable claudin 18.2, optionally wherein the tissue is a tissue other than gastric epithelium.
32. The method of embodiment 30 or 31, wherein the anti-CLDN18.2 antibody therapeutic is zolbetuximab or ASKB589.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound or kit disclosed herein can be used in any of the methods disclosed herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Generation of Anti-CLDN18.2 Antibodies

Female Balb/c mice aged 6-8 weeks were immunized with human claudin 18 and blood was collected one week after three immunizations. Serum titer was measured by indirect ELISA, with OD-blank>0.5 in 1000-fold diluted serum as the qualified standard for titer. Mouse serum with qualified titer was detected by immunohistochemistry (IHC). Mice with histochemical advantage were selected and boosted. At three to four days after boosting, the mouse spleen was removed to perform hybridoma fusion.

Cell culture supernatants of hybridomas were subjected to binding assays by indirect ELISA to screen for positive hybridoma cells. Positive hybridoma clones were cultured, and the supernatants were taken and subjected to ELISA and IHC assays. ELISA and IHC double-positive cells were selected and subjected to one to two rounds of limiting dilution until stable positive cells were obtained. Using the stable positive cells, purified anti-CLDN18.2 monoclonal antibodies were further prepared. An antibody clone DS-3 was selected for further studies. The amino acid sequences of DS-3 are shown in Table 2 (SEQ=SEQ ID NO).

TABLE 2
Amino Acid Sequences of DS-3

| Description | SEQ | Amino Acid Sequence* |
|---|---|---|
| HCDR1 | 1 | SHWVH |
| HCDR2 | 2 | EIDPNNGRTNFNEKFKR |
| HCDR3 | 3 | ARSSLAY |
| LCDR1 | 4 | KSSQSLLDSDGKTYLH |
| LCDR2 | 5 | LVSKLDS |
| LCDR3 | 6 | WQNTHEP |
| VH | 7 | QVQLQQPGAELVKPGASVRLSCKASGYTFSSHWVHWVRQRPGQG |
| | | LEWIGEIDPNNGRTNFNEKFKRKATLTVDKSSSTAYMQYSSLTS |
| | | EDSAVYYCARSSLAYWGQGTLVTVSV |
| VL | 8 | DVVVTQTPLTLSVTIGHPASISCKSSQSLLDSDGKTYLHWFLQR |
| | | PGQSPKRLIYLVSKLDSGVPDRFTGSGSGTDFTLKISRVEAEDL |
| | | GVYYCWQNTHFPWTFGGGTKLEIK |
| Heavy Chain | 9 | QVQLQQPGAELVKPGASVRLSCKASGYTFSSHWVHWVRQRPGQG |
| | | LEWIGEIDPNNGRTNFNEKFKRKATLTVDKSSSTAYMQYSSLTS |
| | | EDSAVYYCARSSLAYWGQGTLVTVSVAKTTAPSVYPLAPVCGDT |
| | | TGSSVTLGCLVKGYFPEPVTLTWNSGSLSSGVHTFPAVLQSDLY |
| | | TLSSSVTVTSSTWPSQSITCNVAHPASSTKVDKKIEPRGPTIKP |
| | | CPPCKCPAPNLLGGPSVFIFPPKIKDVLMISLSPIVTCVVVDVS |
| | | EDDPDVQISWFVNNVEVHTAQTQTHREDYNSTLRVVSALPIQHQ |
| | | DWMSGKEFKCKVNNKDLPAPIERTISKPKGSVRAPQVYVLPPPE |
| | | EEMTKKQVTLTCMVTDEMPEDIYVEWTNNGKTELNYKNTEPVLD |
| | | SDGSYFMYSKLRVEKKNWVERNSYSCSVVHEGLHNHHTTKSFSR |
| | | TPGK |
| Light Chain | 10 | DVVVTQTPLTLSVTIGHPASISCKSSQSLLDSDGKTYLHWFLQR |
| | | PGQSPKRLIYLVSKLDSGVPDRFTGSGSGTDFTLKISRVEAEDL |
| | | GVYYCWQNTHEPWTFGGGTKLEIKRADAAPTVSIFPPSSEQLTS |
| | | GGASVVCFLNNFYPKDINVKWKIDGSERQNGVLNSWTDQDSKDS |
| | | TYSMSSTLTLTKDEYERHNSYTCEATHKTSTSPIVKSFNRNEC |

*The CDRs are defined by the Kabat scheme.

After the amino acid sequences of DS-3 were determined, expression constructs were generated and transfected into suitable mammalian host cells (e.g., CHO cells) to express DS-3 monoclonal antibody.

### Example 2: Determination of Binding Activity of DS-3 Antibody

The antigen-binding activity of DS-3 was evaluated by an ELISA using a substrate coated with CLDN18.2 capture protein (5 μg/ml). The maximum detection concentration of the DS-3 antibody was set to 1.2 μg/ml, and the DS-3 antibody was diluted with a 3-fold gradient. Goat anti-mouse IgG was used as a secondary antibody with a dilution ratio of 1:1500. The data show that the EC₅₀ of binding between DS-3 and CLDN18.2 was 3.59 ng/ml.

### Example 3: Use of DS-3 as an In Vitro Diagnostic Reagent

A claudin 18.2 staining experiment was performed on a Leica Bond™ III fully-automatic immunohistochemistry instrument, and the experimental steps were as follows:
(i) sample pretreatment: sections of a sample to be assayed and quality control slides were placed and baked in a 65±5° C. oven for 30-60 minutes; and the sections were then taken out and cooled to room temperature;
(ii) staining: the cooled sections were stained in accordance with the following protocol:

| Step | Reagent | Time | Temperature |
|---|---|---|---|
| Dewaxing | Dewaxing solution | Default | 72° C. |
| Antigen retrieval | Bond ™ Epitope Retrieval | 10 minutes | 100° C. |
| | Solution 2 | | |
| Deactivation | Peroxide Block | 5 minutes | Room |
| | | | temperature |
| Primary antibody | DS-3 antibody or negative | 15 minutes | Room |
| incubation | control reagent | | temperature |
| Secondary antibody | Secondary antibody | 8 minutes | Room |
| incubation | | | temperature |
| Secondary antibody | Polymer | 8 minutes | Room |
| incubation | | | temperature |
| Color development | DAB Part 1 + DAB Part B | 10 minutes | Room |
| | | | temperature |
| Re-staining with | Hematoxylin | 5 minutes | Room |
| hematoxylin | | | temperature |

(iii) cleaning and mounting: after the staining steps were completed, the sections were taken out from the staining solution, dehydrated and mounted using a Dakewe fully-automatic staining and mounting machine.

Bond™ Dewax Solution is a commercially available product, and the medical device registration number is National Medical Device Registration No. 20140293.

Antigen retrieval buffer (Bond™ Epitope Retrieval Solution 2) is a commercially available product, and the medical device registration number is National Medical Device Preparation 20150327.

Bond™ Wash Solution is a commercially available product, and the medical device registration number is National Medical Device Registration No. 20150492.

The immunochromogenic reagent (Bond™ Polymer Refine Detection) is a commercially available product, and the medical device registration number is National Medical Device Preparation No. 20150558; it includes peroxide blocking solution (Peroxide Block), post-primary antibody reagent (Post Primary, including rabbit anti-mouse IgG), Polymer (containing anti-rabbit Poly-HRP-IgG), DAB Reagent 1 (DAB Part 1), DAB Reagent B (DAB Part B), Hematoxylin.

The negative control reagent used was a mouse IgG2a monoclonal antibody (clone number E5Y6Q, supplier CST) dissolved in antibody diluent.

The composition of the DS-3 antibody in vitro diagnostic reagent includes: 0.1 μg/mL DS-3 antibody, 0.1 M phosphate buffer (a combination of disodium hydrogen phosphate and sodium dihydrogen phosphate), 0.05-0.06 M sodium chloride, a surfactant (specifically 0.5% (v/v) added amount of Triton X-100), 1% (w/v) bovine serum albumin, a bacteriostatic agent (specifically 0.05% (v/v) added amount ProClin™ 300), at pH 7.2-7.5.

### Example 4: Determination of Staining Result Using DS-3 Detection Reagent

The cell staining using the CLDN18.2 antibody reagent was localized in cel membrane and/or cytoplasm. In staining tumor tissue samples, only the staining result of the cell membranes of tumor cells was determined, and cytoplasmic staining was not considered. The entire sample was evaluated: all effective tumor cells on a section should be evaluated and it was required that no less than 100 tumor cells were detected. The standard for determining staining of the CLDN18.2 antibody reagent in tumor tissue: only the staining result of the cell membranes of the tumor cells was determined, and the staining ratio of the tumor cells (TC) was calculated separately for each staining intensity of the cell membrane.

The calculation formula was as follows:

$$TC = \frac{\begin{matrix}Number of tumor cells with Claudin 18.2 \\ cell membrane stained at a certain intensity\end{matrix}}{Total number of tumor cells} \times 100 \%$$

The staining intensities were scored as 0, 1+, 2+, and 3+, and the scores of the staining intensities were determined as follows:
0: no target was stained in tumor cells;
1+: tumor cells showed weak or incomplete cell membrane staining;
2+: tumor cells showed moderate-intensity cell membrane staining; and
3+: tumor cells showed strong cell membrane staining.

51 different samples were taken and the presence of CLDN18.2 in them was determined using the DS-3 detection reagent and a control antibody R detection reagent (antibody 43-14A, AbCam), as described in Example 3. The antibody R detection reagent was a commercially available CLDN18.2 diagnostic reagent and used in accordance with manufacture's instructions.

Where the result was determined to be positive when the cutoff was defined as TC (2+ and 3+)=40%, i.e., TC (2+ and 3+)≥40%, the comparison of the detection results is shown in Table 3. In Table 3, two samples (samples B1786-1 and 221208-18; group B) were determined to be positive using DS-3 and negative using the antibody R detection reagent. The staining results of the two samples were shown in Table 4.

TABLE 3
Comparison of DS-3 Detection Reagent
and Antibody R Detection Reagent*

| | | Antibody R | | |
|---|---|---|---|---|
| | | + | − | Total |
| DS-3 | + | 25 (A) | 2 (B) | 27 (A + B) |
| | − | 0 (C) | 24 (D) | 24 (C + D) |

| | | | |
|---|---|---|---|
| Total | 25 (A + C) | 26 (B + D) | 51 (A + B + C + D) |

*Cutoff, 40%.

TABLE 4
Staining Results with 40% Cutoff

| | DS-3 | | | | Antibody R | | | |
|---|---|---|---|---|---|---|---|---|
| Sample | | | | TC (2+ | | | | TC (2+ |
| No. | 1+ | 2+ | 3+ | and 3+) | 1+ | 2+ | 3+ | and 3+) |
| B1786-1 | 25 | 25 | 20 | 45 | 15 | 25 | 10 | 35 |
| 221208-18 | 20 | 20 | 30 | 50 | 20 | 15 | 15 | 30 |

For sample B1786-1, TC (2+ and 3+) was 45% using the DS-3 detection reagent, and TC (2+ and 3+) was 35% using the antibody R detection reagent. For sample 221208-18, TC (2+ and 3+) was 50% using the DS-3 detection reagent, and TC (2+ and 3+) was 30% using the antibody R detection reagent. While DS-3 and antibody R exhibited similar staining patterns, staining with DS-3 was stronger on some tumor cells, possibly due to different epitopes recognized by two clones or a higher sensitivity of DS-3.

Where the result was determined to be positive when the cutoff was defined as TC (2+ and 3+)=75%, i.e., TC (2+ and 3+)≥75%, the comparison of the detection results is shown in Table 5. The specific results of three samples with differences using the DS-3 detection reagent and the antibody R detection reagent are shown in Table 6.

TABLE 5
Comparison of DS-3 Detection Reagent
and Antibody R Detection Reagent*

| | | Antibody R | | |
|---|---|---|---|---|
| | | + | − | Total |
| DS-3 | + | 6 (A′) | 3 (B′) | 9 (A′ + B′) |
| | − | 0 (C′) | 42 (D′) | 42 (C′ + D′) |

| | | | |
|---|---|---|---|
| Total | 6 (A′ + C′) | 45 (B′ + D′) | 51 (A′ + B′ + C′ + D′) |

*Cutoff, 75%.

TABLE 6
Staining Results with 75% Cutoff

| | DS-3 | | | | Antibody R | | | |
|---|---|---|---|---|---|---|---|---|
| Sample | | | | TC (2+ | | | | TC (2+ |
| No. | 1+ | 2+ | 3+ | and 3+) | 1+ | 2+ | 3+ | and 3+) |
| B753 | 5 | 15 | 60 | 75 | 10 | 20 | 50 | 70 |
| B776 | 5 | 15 | 60 | 75 | 5 | 15 | 50 | 65 |
| B1450-1 | 10 | 15 | 60 | 75 | 5 | 10 | 60 | 70 |

The three samples had relatively small staining differences, with the DS-3 exhibiting higher sensitivity.

### Example 5: Stability of DS-3 Detection Reagent

After three batches (0880812071, 0880812072 and 0880812073) of DS-3 IHC companion diagnostic kits (prepared as described in Example 3) were inspected and qualified, each batch was separately packaged in an appropriate volume and stored in a 37° C. incubator, and taken out on the 0, 3rd, 7th^{h}, and 14th day, respectively. Each of the serial sections of a tissue chip were tested once, the staining results were compared, the stained sections were determined by a professional pathologist, and the determination results were analyzed.

The results show that the localization and intensity of positive signals obtained with the three batches of kits did not show significant changes in four tests over 14 days, and no target positive signals were observed in a negative control reagent. In those three batches of kits, there was no significant difference in the determination results of the same tissue for the same reagent in four tests over 14 days, ΔTC (≥2+)≤10%. In each test over the 14-day period, there was almost no difference in the determination results of the same tissue using the ready-to-use CLDN18.2 mouse monoclonal antibody reagents from the 3 batches of kits, ΔTC (≥2+)≤5%. These results demonstrate the stability of the reagents over a two-week period.

### Example 6: Phase I/II Clinical Study on the Safety, Tolerability, Pharmacokinetics, and Anti-Tumor Activity of ASKB589

In a Phase I/II clinical study on the safety, tolerability, pharmacokinetics and anti-tumor activity of ASKB589 injection in patients with locally advanced or metastatic solid tumors, the DS-3 companion diagnostic reagent was used for patient screening according to the method described in Example 3. The diagnostic test was carried out at an institution designated by the applicant and was used as a basis for selection of patients to be enrolled in the clinical study. Among the 306 patients screened, 51 had negative expression of CLDN18.2, 116 had low CLDN18.2 expression, 33 had medium CLDN18.2 expression, and 106 had high CLDN18.2 expression.

Comparison of DS-3 antibody and other CLDN18.2 antibodies for in vitro diagnosis was carried out. A mouse-derived anti-CLDN18.2 antibody (lot number PW03-33) was obtained through mouse immunization. A rabbit-derived anti-CLDN18.2 antibody (AbCam, clone number EPR19202-244) was also obtained. These two antibodies and DS-3 were compared by immunohistochemical staining.

In human gastric cancer tissue sections, DS-3 showed more significant claudin 18.2 coloration (FIG. 1A) compared to the mouse anti-CLDN18.2 antibody (PW03-33) (FIG. 1B). DS-3 also showed more significant color development of claudin 18.2 (FIG. 1C) compared to the rabbit-derived anti-CLDN18.2 antibody (FIG. 1D).

1. A method of detecting claudin 18.2 (CLDN18.2) in a biopsy obtained from a human, comprising:
contacting the biopsy with an anti-CLDN18.2 antibody or an antigen-binding fragment thereof comprising heavy chain CDR1-3 comprising SEQ ID NOs:1-3, respectively, and light chain CDR1-3 comprising SEQ ID NOs:4-6, respectively; and
detecting the binding of the antibody or antigen-binding fragment to the biopsy, wherein the presence of binding is indicative of the presence of CLDN18.2 in the biopsy.

2. A method of diagnosing cancer in a human, comprising:
contacting a biopsy from the human with an anti-CLDN18.2 antibody or an antigen-binding fragment thereof comprising heavy chain CDR1-3 comprising SEQ ID NOs:1-3, respectively, and light chain CDR1-3 comprising SEQ ID NOs:4-6, respectively; and
detecting the binding of the antibody or antigen-binding fragment to the biopsy, wherein the presence of binding is indicative of the presence of cancer in the human.

3. The method of claim 2, wherein the biopsy is taken from a tissue that does not normally express detectable claudin 18.2.

4. The method of any one of claims 1-3, wherein the antibody or antigen-binding fragment comprises
a heavy chain variable region comprising SEQ ID NO:7 or an amino acid sequence at least 90% identical thereto, and
a light chain variable region comprising SEQ ID NO:8 or an amino acid sequence at least 90% identical thereto.

5. The method of any one of claims 1-4, wherein the antibody or antigen-binding fragment comprises
a heavy chain comprising SEQ ID NO:9 or an amino acid sequence at least 90% identical thereto, and
a light chain comprising SEQ ID NO:10 or an amino acid sequence at least 90% identical thereto.

6. The method of any one of claims 1-5, wherein the antibody or antigen-binding fragment is linked directly or indirectly to a detectable marker.

7. The method of claim 6, wherein the detectable marker is horseradish peroxidase.

8. The method of any one of claims 1-7, wherein the biopsy is obtained from the ear, nose, throat, esophagus, stomach, pancreas, liver, biliary tract, colon, lung, bronchus, breast, or ovary tissue.

9. The method of any one of claims 1-8, wherein the biopsy is sectioned and fixed on a solid support before being contacted with the antibody or antigen-binding fragment, optionally wherein the solid support is a glass slide.

10. A kit comprising an anti-CLDN18.2 antibody or an antigen-binding fragment thereof, wherein the antibody or antigen-binding fragment comprises heavy chain CDR1-3 comprising SEQ ID NOs:1-3, respectively, and light chain CDR1-3 comprising SEQ ID NOs:4-6, respectively.

11. The kit of claim 10, wherein the antibody or antigen-binding fragment comprises
a heavy chain variable region comprising SEQ ID NO:7 or an amino acid sequence at least 90% identical thereto, and
a light chain variable region comprising SEQ ID NO:8 or an amino acid sequence at least 90% identical thereto.

12. The kit of claim 10, wherein the antibody or antigen-binding fragment comprises
a heavy chain comprising SEQ ID NO:9 or an amino acid sequence at least 90% identical thereto, and
a light chain comprising SEQ ID NO:10 or an amino acid sequence at least 90% identical thereto.

13. The kit of any one of claims 10-12, wherein the antibody or antigen-binding fragment is present in a phosphate-buffered solution comprising sodium chloride, a surfactant, bovine serum albumin, and a preservative, with a pH of 7-8.

14. The kit of any one of claims 10-12, wherein the kit comprises
the antibody or antigen-binding fragment in a lyophilized form and
a reconstitution phosphate-buffered solution comprising sodium chloride, a surfactant, bovine serum albumin, and a preservative, with a pH of 7-8.

15. A method of treating cancer in a human in need thereof, comprising:
detecting the presence of CLDN18.2 in a biopsy obtained from the human with a method of any one of claims 1-9, and
treating the patient with an anti-CLDN18.2 antibody therapeutic if CLDN18.2 is found to be present in the biopsy.

16. The method of claim 15, wherein the biopsy is taken from a tissue that does not normally express detectable claudin 18.2.
